(19) United States
(12) Patent Application Publication
(10) Pub. No.: US 2018/0243501 A1
(43) Pub. Date: Aug. 30, 2018

(54) INFUSION SET

(71) Applicant: KOBAYASHI & CO., LTD., Tokyo (JP)

(72) Inventors: Koji Fukuoka, Kobe-shi (JP); Go Kawaoi, Kobe-shi (JP); Imari Endo, Kobe-shi (JP); Masaaki Mitsuhashi, Kobe-shi (JP)

(73) Assignee: KOBAYASHI & CO., LTD., Tokyo (JP)

(21) Appl. No.: 15/758,485

(22) PCT Filed: Sep. 8, 2015
(86) PCT No.: PCT/JP2015/075523

§ 371 Date: Mar. 8, 2018

(51) Int. Cl. A61M 5/14; A61M 39/10; A61M 5/168; A61M 39/28; A61M 39/26

(52) U.S. Cl. CPC A61M 5/1411; A61M 39/10; A61M 5/1407; A61M 5/162; A61M 39/285; A61M 39/26; A61M 2005/1402; A61M 5/16813

(57) ABSTRACT

An infusion set comprising first member(s) constituting primary tubing and second member(s) constituting instant tubing covered by a cap, arranged at an opening configured so as not to allow passage of solid or liquid but so as to allow passage of gas at the interior of the cap, a hydrophobic filter being arranged at a location inward from where the tip of a connector for connection with a three-way stopcock or an intravenous drip needle is inserted at the interior of the cap by way of an insertion port, and a lid at the exterior of said opening for closing said opening. The infusion set makes it possible to carry out priming by means of a simple operation without leakage of liquid; and operations become improved due to the fact that first member(s) and second member(s) can be connected as desired by three-way stopcock(s) 5.

### TECHNICAL FIELD

The present invention relates to an infusion set, and to an infusion set sealed within a package, which, by making it possible for an infusion container to be pierced on a first attempt, permit priming operations to be carried out separately at respective sets of main tubing and secondary to of an infusion set without contamination of areas thereabout, and which, by thereafter making it possible, at any necessary desired timing, for said set of secondary tubing at which the infusion container has been pierced to be made integral with and inseparable from the set of main tubing of the infusion set, permit an infusion to be administered in stable fashion without occurrence of contamination or misidentification of infusion administration procedures.

### BACKGROUND ART

Medical treatments have conventionally been carried out in which therapeutic medications are formulated as infusions which are administered intravenously. Infusions employing therapeutic medications in the form of anticancer agents, nutrients, and the like must typically be administered in high dosages. Furthermore, where a plurality of medications are combined, each infusion must be administered in order, and the total administration dosage is quite high. On the other hand, as administration of infusions continues, because a sudden rise in the concentration of a drug within the blood increases the risk of occurrence of anaphylactic shock, cardiac arrhythmia, and other such side effects, there is a need for physicians and nurses to carefully continue to adjust infusion administration dose while monitoring the condition of the patient. However, carrying out administration by adjusting amount by means of injection is difficult, and as administration time goes on and the number of administrations increases, the patient experiences an increasing amount of bodily pain and is placed under an increasing amount of stress. For this reason, intravenous drip infusion is widely used as a technique for easily and continuously administering infusions to patients intravenously. During intravenous drip infusion, as medical equipment for causing a container having an infusion sealed therewithin to be connected to an intravenous drip needle that has been inserted within a blood vessel of a patient, an infusion set is employed, those in general use conventionally employing soft tubing to link the container having the infusion sealed therewithin with the intravenous drip needle, a mechanism employing a roller clamp and a drip chamber being provided midway along said soft tubing,

Before initiating intravenous drip infusion, physiological saline solution or other such infusion is made to pass through such an infusion set in advance, priming operations being carried out to adequately remove air in advance from the soft tubing, at which time it is necessary to pay adequate attention to ensure that air bubbles originating from such air are not allowed to enter the patient's blood vessel. However, with a conventional infusion set, even where caution is exercised there is a risk that an infusion may leak from the tip of the needle at the downstream-most end of the infusion set during priming, or that an accident could occur in which a hospital room or a hospital wing becomes contaminated, and so there has been a need for a strategy to prevent such accidents.

An infusion set has therefore been proposed that adopts a strategy to reduce the likelihood of leakage of dangerous drugs to the outside environment by splitting the region upstream of the drip chamber into multiple branches to establish two switchable priming flow paths, a shutoff clamp being arranged below the chip chamber and one of the priming flow paths being employed to early out initial priming, and the priming flow path thereafter being switched, the shutoff clamp below the drip chamber being closed, and the other priming flow path thereafter being used to carry out priming of the remaining priming flow path (see Patent Reference No. 1).

However, while it may be true that the risk of contamination during initial priming is relatively low, the structure of the foregoing proposed infusion set is such that it permits leakage of the dangerous drug from below, and so from the standpoint of whether it adequately prevents leakage of dangerous drugs to the outside environment, there has been a need for further ingenuity. Furthermore, because the structure is such that terminal portions of the infusion set are open, operations have been complicated inasmuch as there has been a need to exercise care with respect to procedural discharge of physiological saline solution or other such solution used during priming from said terminal portions, and inasmuch as there has been a need for priming, which itself comprises multiple procedural steps, to be carried out twice, and so forth. In addition, even where adequate care is exercised with respect to procedural discharge of liquid, there is a risk of occurrence of damage to equipment and/or contamination of the hospital room interior as a result of unintentional spillage of the physiological saline solution that is discharged therefrom onto the infusion set, the stand, the equipment used to carry out intravenous drip infusion, and/or the floor of the hospital room.

Furthermore, with a conventional infusion set, where a plurality of anticancer agents were administered, it has been necessary to remove used infusion container(s) from the infusion set and replace these with different infusion container(s), and so it has been entirely possible for contamination to occur due to the small amount of liquid which remains in spike(s) and/or connector portion(s). It therefore being demanded that procedures be carried out with great care, the physician, nurse, and pharmacist responsible for carrying out procedures have been put under an extremely large amount of stress, as a result of which there has been a need for some further strategy.

Infusion sets are therefore in general use in which a plurality of sets of secondary tubing are arranged in advance so as to make it possible to use a mixing region and/or Y-tubing midway along the soft tubing to split it into multiple branches and make it possible to link a plurality of containers having infusions sealed therewithin. Where this is the case, it being necessary to cause this to remain as still as possible to prevent the infusion from leaking outside the blood vessel during intravenous drip infusion, the infusion set is suspended from an IV stand and is used at the patient's bedside, but because at least 3 to 5 hours is required for intravenous drip infusion, and there are cases in which extremely long times on the order of 48 hours may be required, the patient will have no choice but to at some point during the course thereof go to the toilet, eat a meal, or perform some other physiological activity. The patient must therefore, while continuing to receive the intravenous drip infusion with the intravenous drip needle still stuck in his or her arm, or after closing the roller clamp and reducing the drip rate, or temporarily shutting off and interrupting the drip, or the like, move together with the IV stand, linked to which is the infusion set from which the container having the infusion sealed therewithin is suspended.

However, particularly in conventional infusion sets such as those in which a plurality of sets of secondary tubing are arranged in advance, there has been a tendency for balance to become upset and there has been a risk of occurrence of accidents in which tubing at the plurality of sets of secondary tubing that have been arranged in advance gets caught on peripheral equipment and/or the physician, nurse, or pharmacist responsible for carrying out procedures and so forth. Furthermore, even where the patient, physician, nurse, and pharmacist have paid adequate attention with the intention of avoiding situations in which the infusion set tubing itself becomes twisted or tension acts thereon, there has been a tendency toward occurrence of pain at the location where the drip infusion is inserted and: or other such bodily pain on the part of the patient, and in situations where an intravenous drip needle inserted beneath the skin has moved, causing anticancer agent solution to leak outside the blood vessel, or further tension or forcible twisting has acted thereon, there has been an extremely large amount of stress during use, inasmuch as there has been a risk of occurrence of accidents in which the intravenous drip needle could become dislodged from the arm of the patient and/or the like. For this reason, there has been a need for some further strategy.

Furthermore, where a plurality of anticancer agents were administered in the form of infusions, particular attention has been required to avoid mixture of solutions for which mixture is contraindicated and to see that administration is carried out in the correct order. Because the type and number of therapeutic agents are different for every patient, infusion sets must be prepared that are reassembled in correspondence thereto. However, it is often the case that the containers employed for infusions have similar external appearance. For this reason, mixups with respect to the order in which spikes pierce containers, confusion as to which infusion line should be used for different anticancer agent solutions, mistaken order of administration, and other such accidents can easily occur. Efforts have therefore been made to prevent accidents due to human error on the part of physician, nurse, and/or pharmacist who use the infusion set and adjust infusions and anticancer agent solutions by applying labels to the various containers at the time that the infusions are prepared, attaching written warnings, and so forth to clearly specify administration procedure, and to make the various containers recognizable, to establish standard procedures for use specific to each of the various infusion sets with their many different constitutions, to publicize this in advance, and carry out training and so forth, but the situation remains unchanged and misidentification by the operator can still occur, as a result of which there has been a need for improvement.

### PRIOR ART REFERENCES

### Patent References

Patent Reference No. 1: International Patent Application Publication No. 2014/021390

### SUMMARY OF INVENTION

### Problem to be Solved by Invention

A problem to be solved by the present invention is to provide a novel infusion set that makes it possible to, by means of a simpler procedure, easily, conveniently, and definitively implement the priming operations that are carried out prior to use of the infusion set, and in which the risk of occurrence of damage to equipment and contamination of the hospital room interior due to unintentional leakage of liquid from the infusion set while priming operations are being carried out or after priming operations have been carried out is drastically reduced.

Furthermore, another problem to be solved by the present invention is to provide a novel infusion set which, by making it possible for an infusion container to be pierced on a first attempt, permits priming operations to be carried out in definitive fashion separately at a respective first member making up a set of primary tubing and a respective second member making up a set of secondary tubing in an infusion set without contamination of areas thereabout, and which, by thereafter making it possible, at any desired timing, for said set of secondary tubing at which the infusion container has been pierced to be made integral with and inseparable from the set of main tubing of the infusion set, permits an infusion to be administered in stable fashion without occurrence of contamination or misidentification of infusion administration procedures.

Furthermore, another problem to be solved by the present invention is to provide a novel infusion set that makes it possible to prevent bodily pain to patients and/or leakage of liquid that might otherwise occur due to twisting and/or tension acting on the infusion set and/or intravenous drip needle(s) while lowering the risk of occurrence of accidents in which the IV stand from which the infusion set is suspended falls over.

In addition, a problem to be solved by the present invention is to, also in addition to the foregoing, provide a novel infusion set that makes it possible to prevent occurrence of situations in which confusion as to order of use of a plurality of spikes provided at an infusion set when installing infusion container(s) causes error in the order in which infusions are to be administered, or in which infusion(s) containing different drug(s) become mixed up among multiple pieces of infusion tubing when there has been a change in set(s) of infusion(s) at the infusion set, and other accidents such as may occur due to human error, and which facilitates division of labor among workers through adoption of a constitution in which setup of a first member, and a second member making up a set of secondary tubing, can be carried out separately, and that moreover makes it possible to easily establish standard procedures for use.

### Means for Solving Problem

A first means in accordance with the present invention for solving the foregoing problems is an infusion set characterized in that a first member constituting primary tubing in the infusion set has a spike; an optional spike cap; a three-way stopcock; a male connector; a drip chamber; a roller clamp; a connector for connection with an intravenous drip needle; a cap, for installation on the connector for connection with the intravenous drip needle, arranged at which there is an opening not allowing passage therethrough of solid or liquid but allowing passage therethrough of gas, a hydrophobic filter being arranged at a location inward from where a tip of the connector for connection with the intravenous drip needle is inserted at the interior of the cap by way of an insertion port for the connector for connection with said intravenous drip needle, a lid for closing, said opening being arranged at the exterior of said opening; infusion tubing; a shutoff clamp for pressing on and opening and/or closing a flow path within the infusion tubing; and an optional filter bag;

a second member constituting secondary tubing in the infusion set has a spike; an optional spike cap; infusion tubing; a shutoff clamp for pressing on and opening and/or closing a flow path within the infusion tubing; a connector for connection with a three-way stopcock; and a cap, for installation on the connector for connection with the three-way stopcock, arranged at which there is an opening not allowing passage therethrough of solid or liquid but allowing passage therethrough of gas, a hydrophobic filter being arranged at a location inward from where a tip of the connector for connection with the three-way stopcock is inserted at the interior of the cap by way of an insertion port for the connector for connection with said three-way stopcock, a lid for closing said opening being arranged at the exterior of said opening;

wherein, prior to use of the infusion set, the first member constituting the primary tubing and the second member constituting, the secondary tubing in the infusion set are in a mutually separate state; but during use thereof, a secondary tubing branch connector at the three-way stopcock of the first member constituting the primary tubing in the infusion set and a connector that connects with the three-way stopcock at the second member constituting the secondary tubing in the infusion set are connected, causing, the first member constituting the primary tubing and the second member constituting the secondary tubing in the infusion set to be in the form of a single integral unit;

wherein a spike is provided at an upstream-most location in the first member constituting, the primary tubing of the infusion set, said spike being optionally covered with a spike cap, and said spike being connected to one end of infusion tubing equipped with a shutoff clamp for pressing on and opening and/or closing a flow path within infusion tubing, the other end of said infusion tubing being connected to a primary tubing upstream branch connector of a three-way stopcock, and further connected to a primary tubing downstream branch connector of said three-way stopcock there is or are optionally one or more three-way stopcocks, and further connected to the primary tubing downstream branch connector of the three-way stopcock is one end of infusion tubing equipped with a shutoff clamp for pressing on and opening and/or closing an infusion tubing flow path, the other end of said infusion tubing being connected to a drip chamber, said drip chamber being connected to one end of infusion tubing on which a roller clamp is installed, the other end of said infusion tubing being connected to, a primary tubing upstream branch connector of a three-way stopcock, a primary tubing downstream branch connector of said three-way stopcock being connected to one end of infusion tubing, the other end of said infusion tubing being optionally connected to a filter bag and infusion tubing downstream therefrom, and the other end of said infusion tubing being further connected to a primary tubing upstream branch connector of a three-way stopcock, a primary tubing downstream branch connector of said three-way stopcock being connected to one end of infusion tubing the other end of said infusion tubing being connected to a connector for connection with an intravenous drip needle, said connector for connection with an intravenous drip needle being covered with a cap, fur installation on the connector for connection with the intravenous drip needle, arranged at which there is an opening not allowing passage therethrough of solid or liquid but allowing passage therethrough of gas, a hydrophobic filter being arranged at a location inward from where a tip of the connector for connection with the intravenous drip needle is inserted at the interior of the cap by way of an insertion port for the connector for connection with the intravenous drip needle, a lid for closing said opening being arranged at the exterior of said opening; and

wherein a spike is provided at an upstream-most location in the second member constituting the secondary tubing of the infusion set, said spike being optionally covered with a spike cap, and said spike being connected to one end of infusion tubing equipped with a shutoff clamp for pressing on and opening and/or closing a flow path within infusion tubing, the other end of said infusion tubing being connected to a connector for connection with a three-way stopcock, the connector for connection with said three-way stopcock being covered with a cap, for installation on the connector for connection with the three-way stopcock, arranged at which there is an opening not allowing passage therethrough of solid or liquid but allowing passage therethrough of gas, a hydrophobic filter being arranged at a location inward from where a tip of the connector for connection with the three-way stopcock is inserted at the interior of the cap by way of an insertion port for the connector for connection with said three-way stopcock, a lid for closing said opening being arranged at, the exterior of said opening.

A second means in accordance with the present invention for solving the foregoing problems is the infusion set according to claim 1 characterized in that the aforesaid three-way stopcock at which infusion tubing is connected to a secondary tubing branch connector, said infusion tubing, a shutoff clamp linked thereto, and the cap are grouped together as a single group such that one or more thereamong has a location of the same color; and where there are a plurality of three-way stopcocks at which spikes are connected to secondary tubing, groups belonging to adjacent three-way stopcocks are made to have locations of respectively different colors; and said infusion tubing furthermore optionally has colored tape of the same color applied thereon; and any among the three-way stopcock, infusion tubing, shutoff clamp, spike, cap for installation on the spike, and colored tape is furthermore optionally such that a number indicating order of administration is displayed thereon.

A third means in accordance with the present invention for solving the foregoing problems is the infusion set according to claim 2 characterized in that the aforesaid colored infusion set is further sealed within a package at which displayed in mutually offset and partially overlapping fashion on the surface thereof are a plurality of illustrations, the respective illustrations being respectively colored so as to have the same colors as the coloration given to groups within the infusion set sealed therewithin that comprise the aforesaid three-way stopcocks at which infusion tubing is connected to secondary tubing branch connectors, said infusion tubing, shutoff clamps linked thereto, and the caps.

A fourth means in accordance with the present invention for solving the foregoing problems is the infusion set according to claim 3 characterized in that numbers indicating order of administration are further displayed at the aforesaid illustrations.

### Benefit of the Invention

Because an infusion set in accordance with the present invention is provided in the form of an infusion set which is ready for use, there is no need for a pharmacist, physician, or nurse to assemble same in correspondence to each patient who is to be the target of medical treatment. In addition, it is possible to obtain the benefit whereby priming and backpriming are completed in extremely, rapid and simple fashion by causing air within the infusion set to be quickly discharged therefrom not only at primary tubing branch(es) but also at secondary tubing branches), and simultaneously causing same to be filled with physiological saline solution, as a result of only a single operation in which a spike at an upstream-most location in said, infusion set is inserted in an infusion container having sealed therewithin physiological saline solution which is suspended from an IV stand.

Furthermore, no physiological saline solution whatsoever is discharged from cap(s) which is/are installed at spike(s) and connector(s) for connection with intravenous drip needle(s) and at which hydrophobic filter(s) is/are arranged, and so damage to equipment and contamination do not occur. By causing said cap(s) to he provided with clip(s) equipped with catch(es) for flange(s) arranged at spike(s), the risk that cap(s) will become disengaged during priming and backpriming operations is lowered. Because lid(s) provided on cap(s) at which hydrophobic filter(s) is/are arranged are closed following priming and backpriming, physiological saline solution at regions peripheral to hydrophobic filter(s) at cap interior(s) does not spill out from cap insertion port(s). In addition, because the amount of physiological saline solution that is used can be kept to a minimum, such that an adequate remaining amount is retained therewithin, this permits effective use thereof, as there may be no need to replace the infusion container at the time of any medical treatment that may take place thereafter.

Furthermore, in an infusion set in accordance with the present invention, causing, instant tubing of a plurality of three-way stopcocks arranged at various locations to be arranged to the left and the right in balanced fashion will make it possible to achieve a situation in which no abnormal force acts on infusion tubing and/or intravenous drip needle(s), and in which the risk of occurrence of accidents in which intravenous drip needle(s) become detached or there is leakage of liquid is greatly reduced.

In addition, at the infusion set of the present invention, three-way stopcock(s) at which infusion tubing is connected to secondary tubing branch connector(s), said infusion tithing, shutoff clamp(s) linked thereto, and cap(s) are grouped together as a single group such that one or more thereamong is colored with the same color. In particular, where there are a plurality of said three-way stopcocks, groups belonging to adjacent three-way stopcocks are colored so as to be respectively different colors. This makes it possible to obtain the benefit whereby an order of colors is associated with au order of administration of infusions and/or a procedure for using the infusion set, making it possible to facilitate identification of sets of secondary tubing of different groups.

Moreover, at the infusion set of the present invention, by causing the aforesaid colors and the order thereof to be adopted from those in a familiar verse or lyric of a nursery rhyme, and by moreover causing illustration(s) in which colored images associated with the aforesaid verse or lyric of a nursery rhyme based on which these were adopted to be displayed in layered fashion on a package within which the infusion set is sealed, it will be possible to naturally and accurately recognize the order of colors, i.e., the procedure for use of the infusion set and/or the order of administration of infusions, making easy memorization in association with such familiar content possible, and making it possible to more effectively prevent misidentification of administration procedures. In addition, when this is viewed by patients, pharmacists, nurses, and physicians, it will alleviate tension, and will make it possible to simultaneously obtain a psychological effect whereby the atmosphere at sites at which medical treatment is carried out is made more relaxing.

In addition, provision of the infusion set of the present invention will permit smooth progress to be made in standardization of operational procedures for use of the infusion set, and will make it possible to obtain the benefit whereby medical accidents such as mistaken administration or the like occurring as a result of misidentification by a pharmacist, physician, or nurse, or other such human error, are prevented.

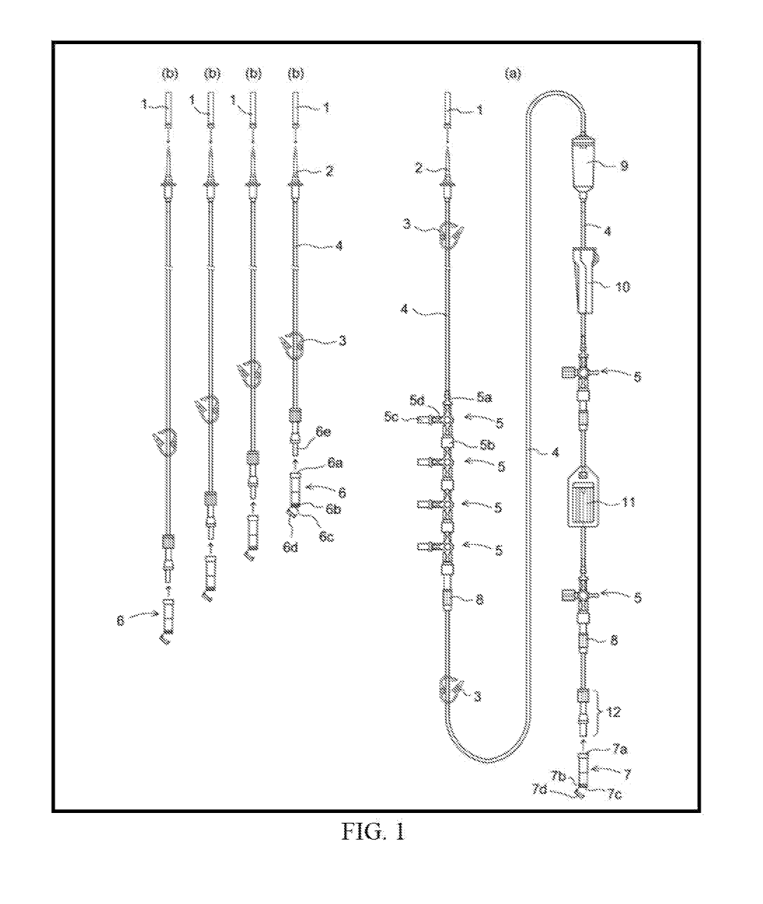

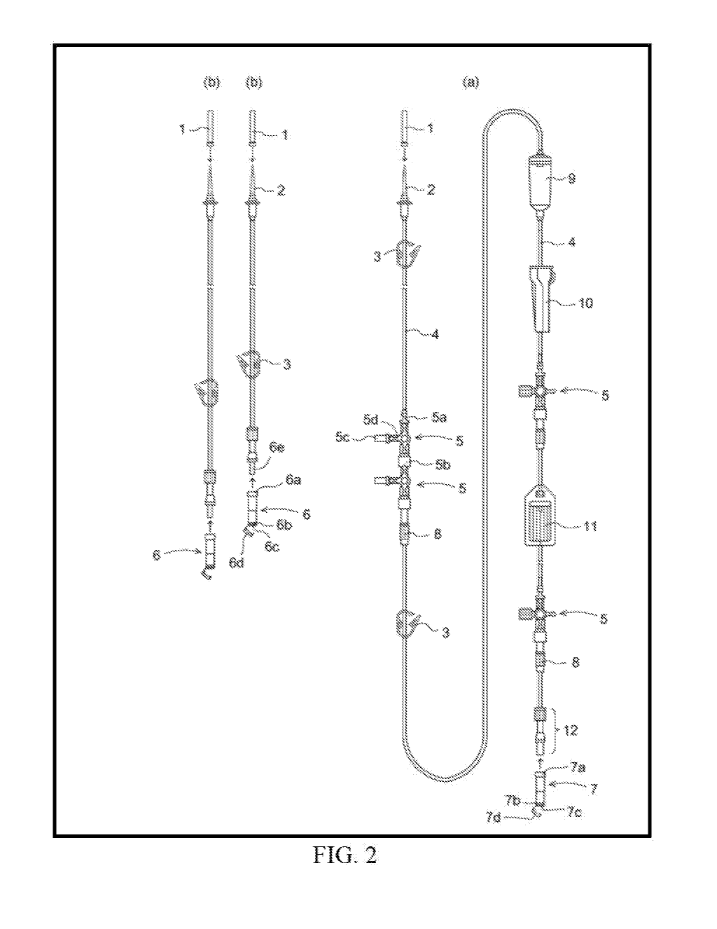

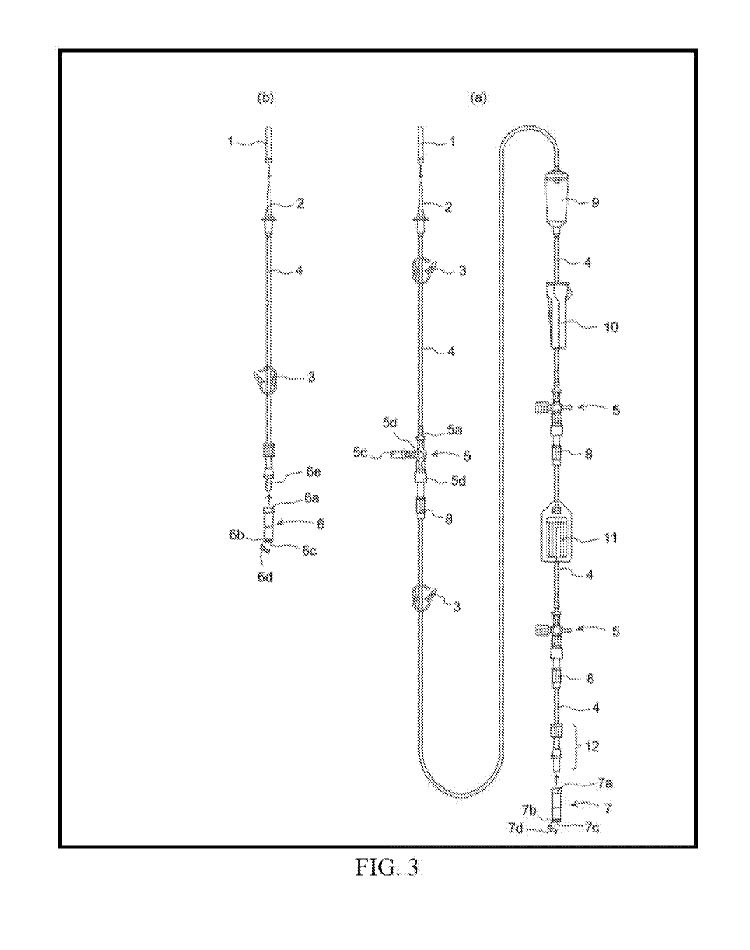

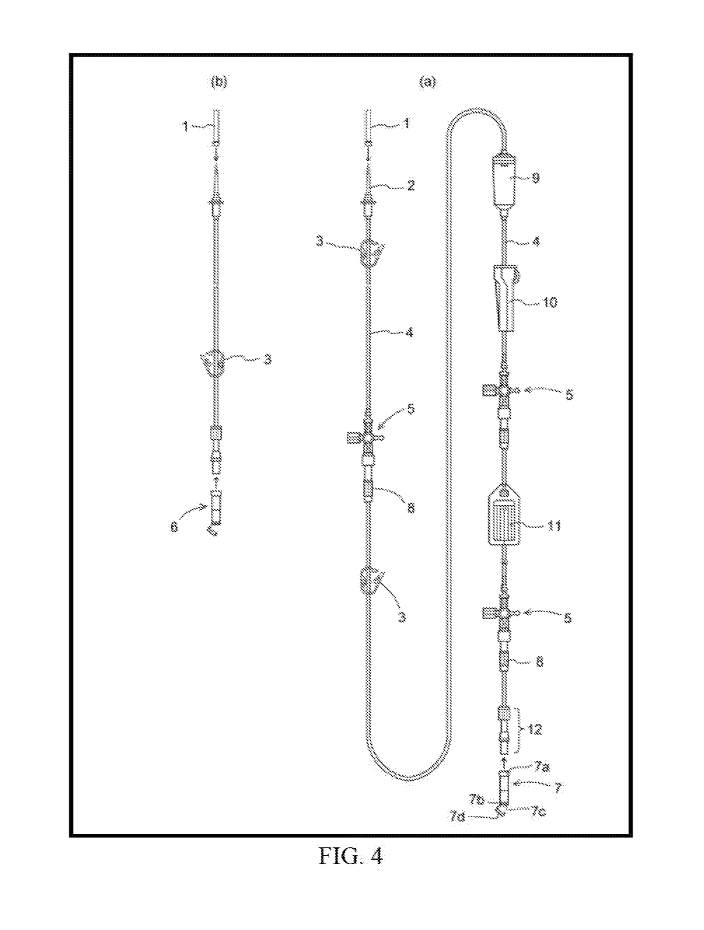

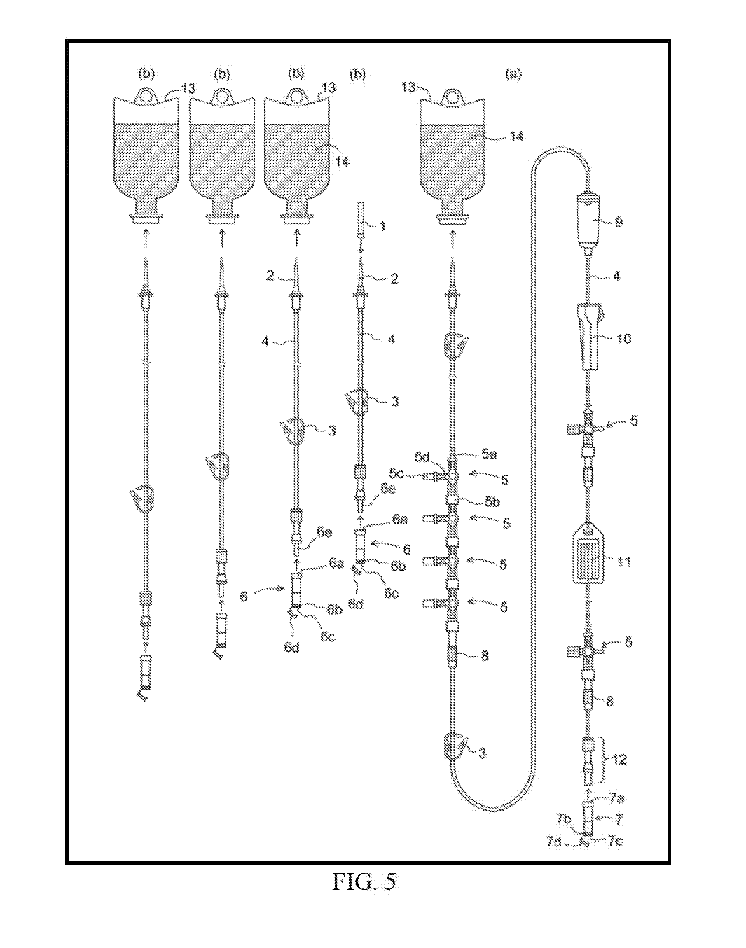

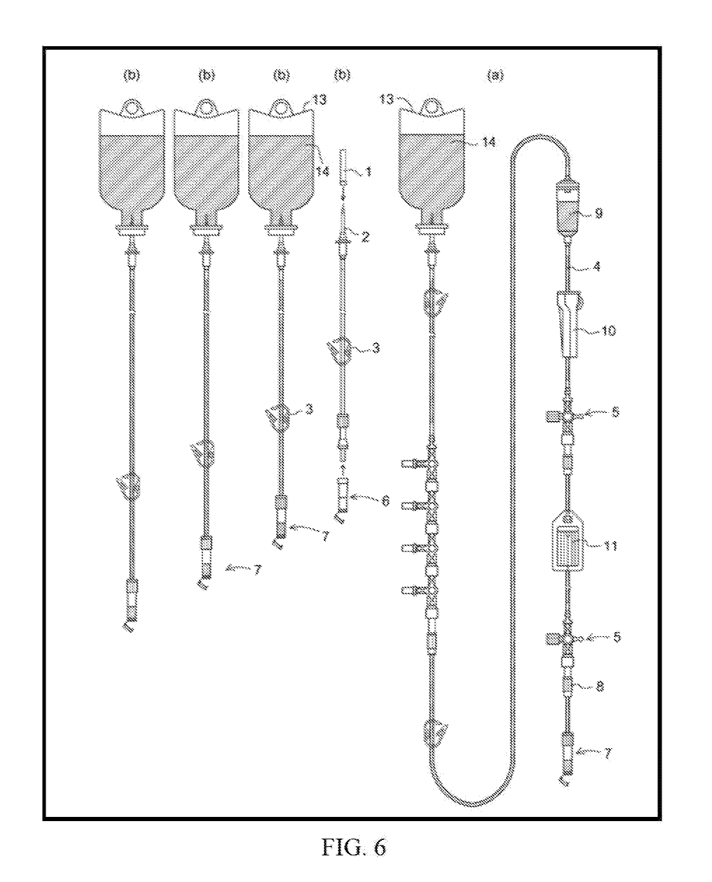

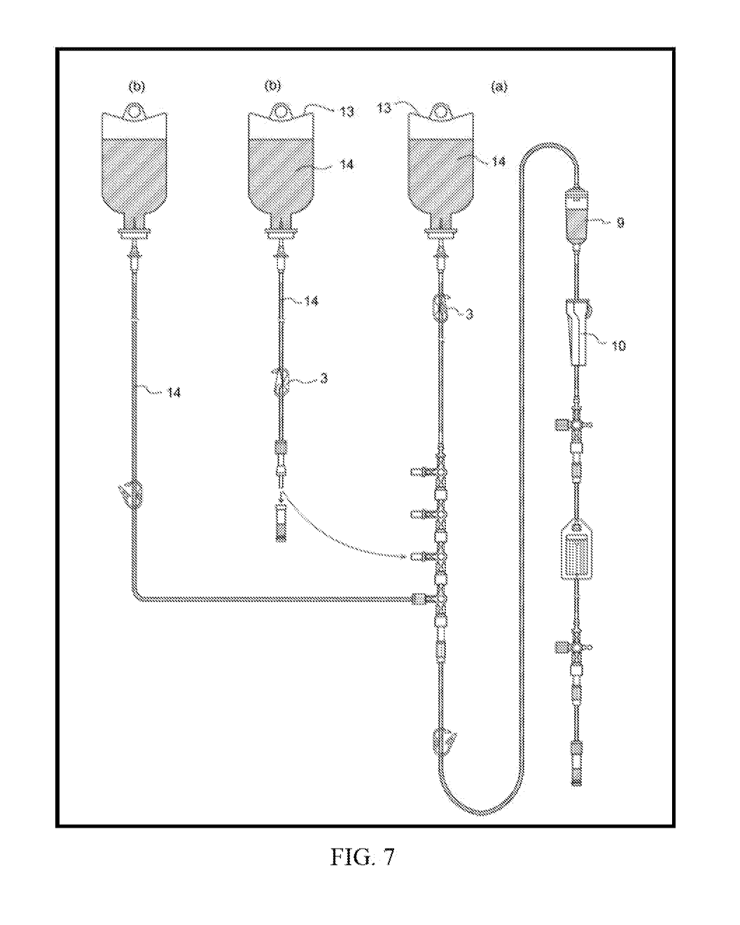

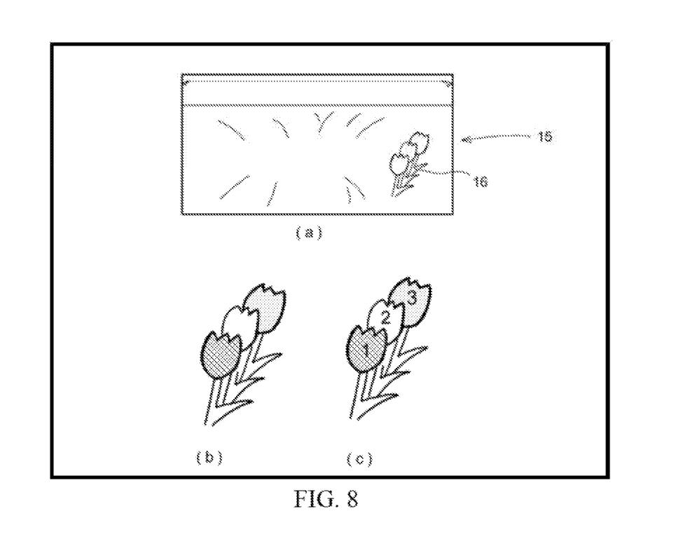

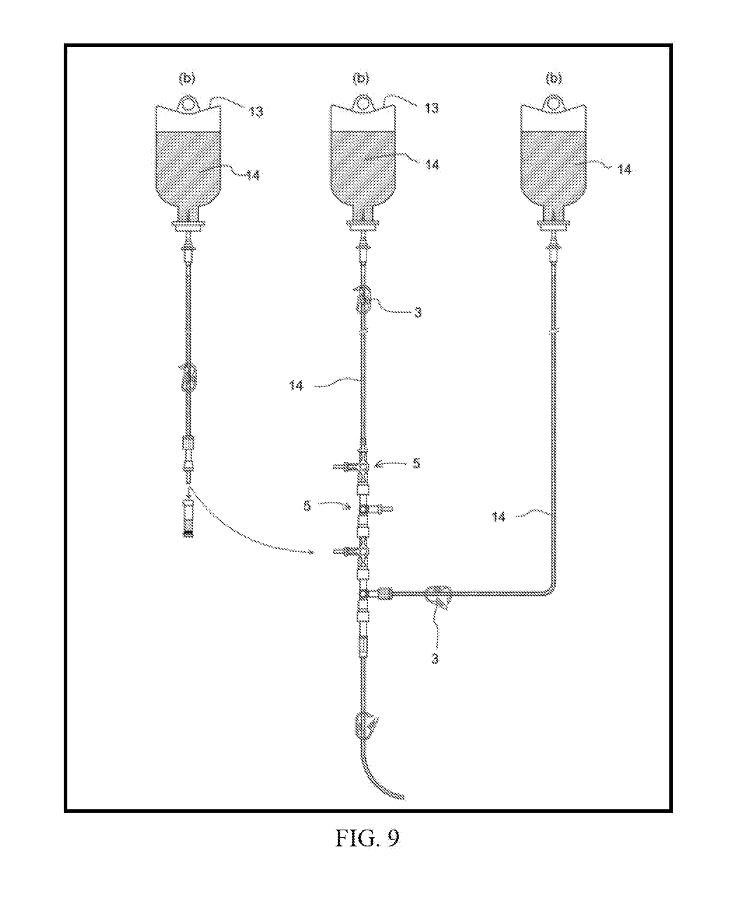

### BRIEF DESCRIPTION OF THE DRAWINGS

[FIG. 1] Explanatory diagram showing an infusion set (in which secondary tubing branch connectors at three-way splitters are needle-less ports) in accordance with the present invention, (a) indicates first member; (b) indicates second members.

[FIG. 2] Explanatory diagram showing an infusion set in accordance with the present invention

[FIG. 3] Explanatory diagram showing an infusion set in accordance with the present invention.

[FIG. 4] Explanatory diagram showing an infusion set in accordance with the present invention.

[FIG. 5] Drawing for explaining a method for carrying out priming in the context of an infusion set in accordance with the present invention.

[FIG. 6] Drawing for explaining a method for carrying out priming in the context of an infusion set in accordance with the present invention.

[FIG. 7] Drawing fur explaining a method for carrying out priming in the context of an infusion set in accordance with the present invention.

[FIG. 8] Drawing for explaining an infusion set in accordance with the present invention that is sealed within a package at which displayed in mutually offset and partially overlapping fashion on the surface thereof are a plurality of illustrations 16. (a) is an explanatory diagram showing the external appearance of a package. (b) is a drawing for explaining the coloration that is applied to the illustrations. (c) is a drawing for explaining numbers indicating order of administration that are further displayed on the illustrations,

[FIG. 9] Explanatory diagram showing an infusion set (in which secondary tubing branch connectors at three-way splitters are needle-less ports and in which connectors for respective sets of instant tubing are arranged so as to be distributed to left and to right) in accordance with the present invention.

### EMBODIMENTS FOR CARRYING OUT THE INVENTION

Below, embodiments for carrying out the present invention are described as appropriate with reference to the drawings.

### Configuration and Constitution of Infusion Set of Present Invention

An infusion set in accordance with the present invention comprises a first member constituting primary tubing in the infusion set, and second member(s) constituting secondary tubing in the infusion set.

The first member constituting primary tubing in the infusion set has spike(s) 2; optional spike cap(s) 1; three-way stopcock(s) 5; male connector(s) 8; drip chamber(s) 9: roller clamps) 10; connector(s) 12 for connection with intravenous drip needle(s); cap(s) 7 for installation on connector(s) for connection with intravenous drip needle(s), arranged at which there is/are opening(s) not allowing passage therethrough of solid(s) or liquid(s) but allowing passage therethrough of gas(es), hydrophobic filter(s) being arranged at location(s) inward from where tip(s) of connector(s) for connection with intravenous drip needle(s) is/are inserted at interior(s) of cap(s) by way of insertion port(s) for connector(s) for connection with intravenous drip needle(s), licks) for closing said opening(s) being arranged at exterior(s) of said opening(s); infusion tubing 4; shutoff clamp(s) 3 for pressing on and opening and/or closing flow paths) within infusion tubing; and optional filter bag(s) 11 ((a) at FIGS. 1 through 4).

Furthermore, the second member(s) constituting secondary tubing in the infusion set have spike(s) 2; optional spike cap(s) 1; infusion tubing 4; shutoff clamp(s) 3 for pressing on and opening and/or closing flow path(s) within infusion tubing; connector(s) 6e for connection with three-way stopcock(s); and cap(s) 6 for installation on connector(s) for connection with three-way stopcock(s), arranged at which there is/are opening(s) not allowing passage therethrough of solid(s) or liquid(s) but allowing passage therethrough of gas(es), hydrophobic filter(s) being arranged at location(s) inward from where tip(s) of connector(s) for connection with three-way stopcock(s) is/are inserted at interior(s) of cap(s) by way of insertion port(s) for connector(s) connected to said three-way stopcock(s), lid(s) for closing said opening(s) being arranged at exterior(s) of said opening(s) ((b) at FIGS. 1 through 4).

In addition, the infusion set is such that, prior to use thereof, the first member constituting primary tubing and the second member(s) constituting secondary tubing in the infusion set are in a mutually separate state. For this reason, it is possible for the first member constituting primary tubing and the second member(s) constituting secondary tubing in the infusion set to remain in a mutually separate state as the interiors thereof are filled with physiological saline solution or other such solution for the purpose of causing these to he made to assume a primed state. In addition, during use, when infusion container(s) are connected to the infusion set, secondary tubing branch connector(s) for three-way stopcock(s) at the first member and connector(s) connected to three-way stopcock(s) at the second member(s) are connected, causing the first member constituting primary tubing and the second member(s) constituting secondary tubing in the infusion set to be in the form of a single integral unit. So, as to prevent leakage of solution during use as an infusion set, it is preferred that connections therebetween be constituted in such fashion as to make it impossible for these to again become separate.

In addition, the respective members are connected so as to have constitution(s) as indicated below. That is, the first member constituting primary tubing in an infusion set in accordance with the present invention is equipped with spike(s) 2 at upstream-most location(s) thereat, said spike(s) being optionally covered with spike cap(s), said spike(s) being connected to one end of infusion tubing 4 equipped with shutoff clamp(s) 3 for pressing on and opening and/or closing flow path(s) within infusion tubing, the other end of said infusion tubing 4 being connected to primary tubing upstream branch connector(s) 5a of three-way stopcocks) 5. In addition, connected to primary tubing downstream branch connector(s) of said three-way stopcock(s) there is/are optionally one or more three-way stopcock(s) 5. In addition, male connector(s) 8 is are connected to primary tubing downstream branch connector(s) 5b of the aforesaid three-way stopcock(s) 5 disposed at downstream location(s), one end of infusion tubing 4 equipped with shutoff clamp(s) 3 for pressing on and opening and/or closing flow path(s) within infusion tubing being further connected thereto, drip chamber(s) 9 being connected to the other end of said infusion tubing 4. In addition, said drip chamber(s) 9 is/are connected to one end of infusion tubing 4 on which roller clamp(s) 10 is/are installed, the other end of said infusion tubing 4 being connected to primary tubing upstream branch connector(s) 5a of three-way stopcock(s) 5. In addition, primary tubing downstream branch connector(s) 5b of said three-way stopcock(s) 5 is are connected to male connector(s) 8, one end of infusion tubing 4 being further connected thereto, and optionally filter bag(s) 11 and infusion tubing 4 downstream therefrom being connected to the other end of said infusion tubing 4. Moreover, the other end of said infusion tubing 4 is, connected to primary tubing upstream branch connector(s) 5a of three-way stopcock(s) 5. In addition, the constitution of this is in a state such that primary tubing downstream branch connector(s) 5b of said three-way stopcock(s) 5 is/are connected to male connector(s) 8, one end of infusion tubing 4 being further connected thereto, and the other end of said infusion tubing 4 being connected to connector(s) 12 for connection with intravenous drip needle(s) covered with cap(s) 7 for installation on connector(s) for connection with intravenous drip needle(s), arranged at which there is/are openings) not permitting passage therethrough of solid(s) or liquid(s) but permitting passage therethrough of gas(es), hydrophobic filter(s) being arranged at location(s) inward from where tip(s) of connector(s) for connection with intravenous drip needle(s) is/are inserted at interior(s) of cap(s) by way of insertion port(s) for connector(s) for connection with intravenous drip needle(s), and lid(s) for closing said openings) at exterior(s) of said openings). As the foregoing three-way stopcock(s), note that three-way stopcock(s) at which secondary tubing branch connector(s) is/are needle-less port(s) may be employed, and/or those having general-purpose three-way stopcocks) may be employed (FIG. 4). Because the first member and the second member are linked following priming, it is preferred that three-way stopcock(s) at which secondary tubing branch connector(s) is/are needle-less port(s) which facilitate procedures such as those in which linked location(s) is/are wiped off and cleaned be employed Where general-purpose three-way stopcock(s) are employed, it is preferred that these be in a state such that they are covered with cap(s) until such time as the second member(s) are connected to secondary tubing branch connector(s).

Furthermore, the second member(s) constituting secondary tubing in the infusion set in accordance with the present invention are equipped with spike(s) 2 at upstream-most location(s) therein. In addition, said spike(s) are optionally covered with spike cap(s) 1. Moreover, said spike(s) are connected to one end of infusion tubing 4 equipped with shutoff clamp(s) 3 for pressing on and opening and/or closing flow path(s) within infusion tubing. In addition, the other end of said infusion tubing 4 is connected to connector(s) 6e for connection with three-way stopcock(s). The constitution of this is in a state such that said connector(s) for connection with three-way stopcock(s) are covered with cap(s) 6 for installation on connector(s) for connection with three-way stopcock(s), arranged at which there is/are openings) not permitting passage therethrough of solid(s) or liquid(s) but permitting passage therethrough of gas(es), hydrophobic filter(s) being arranged location(s) inward from where tip(s) of connector(s) for connection with three-way stopcock(s) is/are inserted at interior(s) of cap(s) by way of insertion port(s) for said connector(s) for connection with three-way stopcock(s), and lid(s) for closing said opening(s) at exterior(s) of said opening(s).

These first member and second member(s) are such that the respective members are connected in advance so that the first member and the second member(s) are each respectively in the form of a single integral unit, these being provided to a user as an infusion set capable of instant use when the first member and the second member(s) are primed and these are connected.

Where it is necessary to remove debris or the like such as may be produced when spike(s) 2 pierce infusion container(s) 13 and/or other such solids such as may be present within infusion(s), the aforesaid filter bag(s) 11 is/are incorporated in the infusion set, and the respective members are connected in advance in the form of single integral unit(s) provided as an infusion set capable of instant use. In situations where a formulation contraindicated for use with filter bags due to the possibility that it may cause clogging is employed, in situations where no solids are present within the infusion, and in situations where a formulation having high viscosity for which it would be difficult to ensure achievement of adequate drip rate of the infusion at the time of intravenous drip infusion is employed, a constitution is employed in which said aforesaid filter bag(s) 11 is/are omitted, and the respective members are connected in advance in the form of single integral unit(s) provided as an infusion set capable of instant use.

In accordance with the infusion set of the present invention, three-way stopcock(s) 5 at which infusion tubing 4 is connected to secondary tubing branch connector(s) 5c, said infusion tubing 4, shutoff clamp(s) 3 linked thereto, spike(s) 2, and cap(s) 1, 6, 7 may be grouped together as a single group such that one or more thereamong is made to have location(s) of the same color. Moreover, where said infusion set has a plurality of three-way stopcocks 5 at which spikes 2 are connected to secondary tubing, groups belonging to adjacent three-way stopcocks 5 may be made to have location(s) of respectively different colors. Where coloring is carried out, respective groups are colored so as to be of different colors in correspondence to the order in which infusion(s) is/are to be administered so as to permit identification thereof.

However, despite attempts to memorize whatever unremarkable order of colors may have been established, where there is no deep meaning attached to the colors and/or order thereof, there is a risk that the colors and/or order thereof will be misidentified, for which reason particularly careful attention is demanded of nurses and physicians.

To avoid risk of misidentification, it is preferred that colors and an order thereof such as appear in a nursery rhyme, lyric, or verse that many people will know, having been familiar therewith from a young age, he employed as the colors and order thereof for identifying the aforesaid respective groups. For example, where provided in Japan, the colors and order indicated by “red, white, yellow” appearing in lyrics about tulips in the nursery rhyme might be employed; or where provided in an English-speaking region, the colors and order indicated by “red, blue, white” in the verse which goes roses are red, violets are blue, sugar is sweet, and so are you that comes from Mother Goose might be employed, respective groups being colored with different colors in order of use so as to permit identification thereof.

By thus causing an infusion set to employ coloration constituted so as to permit colors and order thereof to he memorized in association with lyrics, verses, and/or other such familiar content, as compared with memorization of an unremarkable order of colors established in the context of standardized administration procedures, nurses and physicians can more naturally and accurately recognize order of colors, i.e., procedures for use of the infusion set and/or order of administration of infusions, and as easy memorization in association with such familiar content is facilitated, it is possible to more effectively prevent misidentification of administration procedures. Moreover, this will permit smooth prowess to be made in standardization of operational procedures for preventing misidentification of administration procedures at sites where many pharmacists, nurses, and physicians are engaged, in medical treatment.

In addition to the aforesaid coloration, it is possible by further optionally causing any among three-way stopcock(s), infusion tubing, shutoff clamp(s), spike(s), cap(s), and/or colored tape(s) to be in a state in which number(s) indicating order(s) of administration are displayed thereon, to even more effectively prevent misidentification of standardized administration procedures.

An infusion set provided with coloration and optionally with display of numbers indicating order of administration as described above is sealed within a pouch or other such package 15 and is provided to a site at which medical treatment is carried out. Here, to even further increase the effect whereby misidentification of standardized administration procedures may be prevented as a result of coloration and display of number(s) indicating order(s) of administration, the infusion set may be sealed within a package at which displayed in mutually offset and partially overlapping fashion on the surface thereof are a plurality of illustrations ((a) at FIG. 11). It is preferred that the infusion set be such that the foregoing illustrations are respectively colored so as to have the same colors as the coloration given to groups within the infusion set comprising the aforesaid three-way stopcocks at which infusion tubing is connected to secondary tubing branch connectors, said infusion tubing, shutoff clamps linked thereto, spikes, and caps for installation on spikes, and so as be colored in order from the frontmost illustration to the backmost illustration in correspondence to the order of use ((b) at FIG. 11). It is more preferred that the aforesaid colored illustrations be such that numbers indicating order of administration be displayed thereon in ascending order from the frontmost illustration to the backmost illustration ((c) at FIG. 11).

From frontmost to backmost, the illustrations on the aforesaid package should respectively employ, and be respectively colored, in order of appearance of images associated with a familiar verse or lyric of the nursery rhyme based on which coloration of the infusion set is carried out. By thus providing an infusion set sealed within a package displaying illustrations, nurses and physicians can, merely by looking at the package before or after the package is opened, extremely naturally and without difficulty gasp the colors and the order thereof, i.e., the order of administration, memorized in association with lyrics, verses, and/or other such familiar content making it possible to more effectively prevent misidentification of standardized administration procedures. Moreover, by causing numbers indicating order of administration to be, displayed at the respective colored illustrations, the order of use of the respective sets of infusions in the infusion set can be linked to colors and definitively recognized, making it possible to greatly reduce the likelihood of occurrence of human error.

Moreover, an infusion set sealed within a package displaying illustrations such as images associated with the aforesaid familiar verse or lyric of a nursery rhyme, when viewed by patients, nurses, and physicians will also alleviate tension, making it possible to simultaneously obtain a psychological effect whereby the atmosphere at sites at which medical treatment is carried out is made more relaxing.

### Materials

There is no particular limitation with regard to the materials that may be employed for formation of the members that make up the infusion set of the present invention, it being possible to use materials such as are ordinarily employed in the context of members for infusion sets and medical equipment; for example, Nylon, polycarbonate, polypropylene, polystyrene, and/or other such resin materials and/or stainless steel and/or other such metals may be employed, it being possible to employ polyolefinic resins and/or other such materials suitable for soil tubing at the infusion tubing. Depending on the type of drug, e.g., anticancer agent, used, it may be the case that plasticizer will leach out therefrom. Resin material(s) employed for formation of the members that make up the infusion set of the present invention therefore employ substance(s) free of polyvinyl chloride (PVC) and/or PVC that does not contain DEHP ethylhexyl phthalate), or else Nylon and/or polycarbonate is employed. Hydrophobic filter(s) may employ polytetrafluoroethylene (PTFE), polyethylene (PE), polyolefin, polypropylene, polyethylene, polyvinylidene fluoride (PVDF), nitrocellulose, and/or the like, it being preferred that polyethylene (PE) and/or polytetrafluoroethylene (PTFE) be employed therefor. Any of the various foregoing resin materials may be employed in colored form. Furthermore, stainless steel and/or other metals may be employed in a form in which the surface thereof has undergone coloration treatment, in which case it is, preferred that material(s) which have undergone coloration treatment that is highly anticorrosive be employed.

### Manufacturing Operations

The infusion set of the present invention is manufactured in such, fashion that the respective members making up the first member and the second member(s) are definitively connected so as to form an infusion set in the form of single integral unit(s). There is no particular limitation with regard to the method for definitively connecting the respective members, it being possible to use methods such as are ordinarily employed as methods for obtaining an infusion set or medical equipment in the form of single integral unit(s); for example, adhesive operations employing adhesives ordinarily used for infusion sets or other such medical equipment, fusing operations that make use of heat, ultrasonic waves, or the like, and/or other such techniques may be utilized. By providing the infusion set in the form of single integral unit(s), the risk that joined parts will become separated is eliminated, making it possible to more definitively prevent medical accidents such as damage to equipment and/or contamination of the hospital room interior due to occurrence of unintentional leakage of liquid.

Moreover, so as to permit immediate commencement of priming operations after the package has been opened and the infusion set has been removed therefrom, an infusion set in accordance with the present invention may be provided in presterilized form. There is no particular limitation with regard to the method for sterilization of the infusion set, it being possible to use methods such as are ordinarily employed as methods for sterilization of infusion sets and/or medical equipment for example, methods which include ethylene oxide gas sterilization, γ irradiation sterilization, e-beam sterilization, radiation sterilization, ultraviolet irradiation sterilization, hydrogen peroxide sterilization, and ethanol sterilization may be employed. In addition, as said sterilization method, it is preferred based on considerations which include ease of manufacturing and cost reduction that ethylene oxide gas sterilization, e-beam sterilization, and/or γ irradiation sterilization be employed. It is preferred that e-beam sterilization be carried out to such a degree as will not cause degradation of the infusion set, and it is preferred that the irradiative energy during γ irradiation sterilization be within a range that is up to on the order of 5 kGy to 30 kGy so as to cause sterilization to be carried out to such a degree as will not cause degradation of the in set.

### Procedure for Using Infusion Set of Present Invention

At the infusion set of the present invention, priming operations are first respectively carried out at the first member and the second member(s) so as to remove air from the interior of the infusion set and cause it to be filled with physiological saline solution or other such solution.

Package 15 within which the infusion set is sealed is opened, and investigation is carried out to make sure that, at the first member and at the second member(s) for instant use in the infusion set, cap(s) 6 for installation on connector(s) for connection with three-way stopcock(s) provided with lid(s) and cap(s) 7 for installation on connector(s) for connection with intravenous drip needle(s) provided with lid(s) are definitively installed thereon, and to make sure that lid(s) 6d, 7d of the respective caps are open. And at the second member(s) for instant use as well, investigation with respect to the state(s) of cap(s) is carried out in similar fashion. Furthermore, investigation is carried out to make sure that all shutoff clamp(s) 3 are in their open state(s).

Where three-way stopcock(s) constituting links) to the second member(s) have lever(s) 5d, investigation is carried out to make sure that these are positioned so as to permit flow to/from all three branches. Furthermore, where three-way stopcock(s) at which there is no spike or infusion tubing connected at secondary tubing branch(es) for use as emergency port(s) have lever(s) 5d, investigation is carried out to make sure that these are in position(s) permitting flow to/from primary tubing upstream branch(es) and primary tubing downstream branch(es) but not permitting flow to/from secondary tubing branch(es). Furthermore, investigation is carried out to make sure that roller clamp(s) 3 are in their open state(s) (FIG. 5).

Next, infusion containers 13 having sealed therewithin physiological saline solution 14 for use during priming of the first member and the second member(s) in an infusion set in accordance with the present invention are respectively suspended from IV stand(s) or other such stand(s). Next, cap(s) 1 for installation on spike(s) installed on spike(s) disposed, at upstream-most location(s) at the first member and the second member(s) in an infusion set in accordance with the present invention is/are removed. In addition, spike 2 of the first member is used to pierce the infusion container 13 for priming the first member, spike 2 of the second member is used to pierce the infusion container 13 for priming the second member, and respective priming operations are initiated (FIG. 5 and FIG. 6). At the first member, in the event that secondary tubing branch connector(s) employ three-way stopcock(s) having needle-less port(s), such three-way stopcock(s) are secured by means of securing hardware with which the stand(s) are equipped. Furthermore, where IV stand(s) are equipped with stay rod(s) for securing drip chamber(s) 9 or other such securing hardware, these may be used to further secure the foregoing. Note that it is preferred even where securing is not carried out during priming that securing hardware he used to secure three-way stopcock(s) 5 and drip chamber(s) 9 when initiating intravenous drip infusion, to prevent situations in which the entire weight of the infusion set is borne by intravenous drip needle(s) and connector(s) 12 for connection with intravenous drip needle(s), so that region(s) at which intravenous drip needle(s) are inserted are not placed under stress.

Physiological saline solution 14 flows into infusion tubing 4 from infusion container(s) 13 placed on spike(s) 2 disposed at upstream-most location(s), and priming operations commence. As physiological saline solution 14 displaces air within infusion tubing 14 of the first member, it flows from primary tubing upstream branch connector 5a of the three-way stopcock 5 which is disposed downstream therefrom into three-way stopcock 5. Because lever 5d of said three-way stopcock 5 is positioned so as to permit flow to/from all three branches, as physiological saline solution 14 flowing thereinto displaces air within three-way stopcock 5, it further flows into primary tubing downstream branch connector 5b, and further causes air within infusion tubing 4 which is connected thereto to be displaced toward a location downstream therefrom. In addition, as physiological saline solution 14 which has flowed thereinto displaces air from within infusion tubing 4 further connected thereto to a location downstream therefrom, it flows into the interior of three-way stopcock 5 and filter bag 11 which are connected at the far end thereof, and as it displaces air from within infusion tubing 4 further connected thereto to a location downstream therefrom, it flows therealong to flow into connector 12 for connection with an intravenous drip needle.

After flowing into connector 12 for connection with an intravenous drip needle, physiological saline solution 14, as it displaces the air therewithin to a location downstream therefrom, flows from the tip of connector 12 for connection with an intravenous drip needle into the interior of cap 7 which is for installation on a connector for connection with an intravenous drip needle, arranged at which there is an opening not permitting passage therethrough of liquid(s) or solid(s) but permitting passage therethrough of gas(es) at the interior of the cap, a hydrophobic filter being arranged at a location inward from where the tip of the connector for connection with an intravenous drip needle is inserted at the interior of the cap by way of the insertion port for the connector for connection with an intravenous drip needle, and a lid for closing said opening at the exterior of said opening. In addition, physiological saline solution 14 which has flowed thereinto causes air that had been present in the region peripheral thereto to further pass through hydrophobic filter 7b and to be displaced to the exterior by way of cap opening 7c, as a result of which the region peripheral thereto becomes filled with physiological saline solution 14. Here, hydrophobic filter 7b does not permit passage therethrough of physiological saline solution 14. Furthermore, air remains in the portion toward the base from the tip of connector 12 for connection with an intravenous drip needle from which physiological saline solution 14 flows at the interior of cap 7.

In similar fashion as at the first member, at the second member as well, physiological saline solution 14 flows into infusion tubing 4 from infusion container 13 placed on spike 2 disposed at the upstream-most location, and priming operations commence. As physiological saline solution 14 displaces air within infusion tubing 14 of the second member, it displaces air from within infusion tubing 4 connected thereto to a location downstream therefrom, and as it does so it flows therealong to flow into connector 6e for connection with a three-way stopcock. In addition, physiological saline solution 14 which has flowed thereinto causes air that had been present in the region peripheral thereto to further pass Through hydrophobic filter 6b and to be displaced to the exterior by way of cap opening 6c, as a result of which the region peripheral thereto becomes filled with physiological saline solution 14. Here, hydrophobic filter 6b does not permit passage therethrough of physiological saline solution 14. Furthermore, within cap 6 air remains in the portion that is more toward the base than, the tip of connector 12 for connection with a three-way stopcock from which physiological saline solution 14 flows.

hi this way, at an infusion set in accordance with the present invention, priming is completed by causing air within each the first member and the second member of said infusion set to be quickly discharged therefrom and simultaneously causing said infusion set to be filled with physiological saline solution as a result of only a single operation in which “spike(s) 2 disposed at upstream-most location(s) in said infusion set are inserted in infusion container(s) 13 which have sealed therewithin physiological saline solution 14 for use during priming and which are suspended from IV stand(s)” (FIG. 6). In addition, regardless of which of the foregoing situations applies, because hydrophobic filter(s) 6b, 7b is,/are present at cap(s) 6 for installation on connector(s) for connection with three-way stopcock(s) and at cap(s) 7 installed on connector(s) for connection with intravenous drip needle(s), there will be no occurrence of leakage of liquid even if lid(s) is/are open.

During the aforesaid priming operations, because hydrophobic filter(s) 6b, 7b is/tare present, there will be absolutely no discharge of unwanted liquid, and there will be no occurrence of damage to equipment and/or contamination of the hospital room interior. Moreover, because the amount of physiological saline solution sealed within infusion container(s) connected to spike(s) at upstream-most location(s) that is used can he kept to a minimum, such that an adequate remaining amount is retained therewithin, this permits effective use thereof, as there may be no need to replace the infusion container(s) at the time of medical treatment that may take place thereafter. Furthermore, because an infusion set in accordance with the present invention is assembled in advance in the form of single integral unit(s), there being no occurrence of unintentional leakage of liquid during priming and backpriming operations, there will be no occurrence of damage to equipment and/or contamination of the hospital room interior.

After completion of priming and backpriming, lid(s) 6d, 7d at cap(s) for installation on connector(s) for connection with three-way stopcock(s) and at cap(s) for installation on connector(s) for connection with intravenous drip needle(s) are closed. In addition, shutoff clamp(s) 3 disposed at respective set(s) of infusion tubing 4 is are pinched together, and roller clamp(s) 10 at the first member is/are moreover moved to their closed position(s), to close off the respective set(s) of infusion tubing 4. Furthermore, lever(s) 5d at three-way stopcock(s) 5 is/are moved to position(s) corresponding to the intravenous drip infusion(s) to be initiated. Investigation is carried out to make sure that there is no leakage of physiological saline solution 14.

Next, the interior of an infusion container connected to the second member is filled with anticancer agent and this is agitated in homogeneous fashion to prepare a therapeutic solution.

When preparatory procedures at the first member and the second member which will be used have been completed, those members are moved to the patient's bedside. At the patient's bedside, connector 6e for connection with three-way stopcock at the second member is connected and secured to secondary tubing branch connector 5c at a three-way stopcock of the first member. Where items divided into groups by color and/or number are used at the respective members, connection is carried out after first investigating to make sure that the secondary tubing branch connector 5c at the three-way stopcock and the connector 6e for connection with the three-way stopcock are of matching color and/or number group. This will make it possible to avoid mistaken connections that might otherwise occur due to human error. Note that where secondary tubing branch connector 5c at the three-way stopcock and connector 6e for connection with the three-way stopcock are equipped with securing fixtures for securing these investigation is carried out to make sure that these are definitively secured. Prior to connection, note that connector 6e for connection with three-way stopcock is wiped off or other such procedures are carried out to cause it to be cleaned as appropriate in advance.

Where a plurality of infusion sets are used, there is a need to prevent accidents in which intravenous drip needle(s) become detached or there is leakage of liquid from region(s) at which intravenous drip needle(s) are inserted due to, application of torsional forces on infusion tubing or accidents in which IV stand(s) fall over due to changes in the distribution of weight. Furthermore, where an anticancer agent or the like is administered by intravenous drip infusion, because particularly long times are required, there is a need to accommodate the fact that the patient will move about and to implement measures to prevent accidents of the same type as described above. Where the present infusion set is such that a plurality of second members are connected, it is therefore preferred that orientations of secondary tubing branch connectors at the plurality of three-way stopcocks disposed therein be such that those which are employed are distributed to both the effect and the right or that some other such strategy be utilized to cause the balance in the distribution of weight at the infusion set to be maintained (FIG. 9). By so doing, the risk that an abnormal force will act on the intravenous drip needle, and the risk of occurrence of accidents in which the intravenous drip needle becomes detached or there is leakage of liquid, will be greatly reduced.

Where an infusion set in accordance with the present invention is used to administer an intravenous drip infusion, the infusion may be allowed to drip under the influence of gravity with drip rate being adjusted by drip chamber 9 and roller clamp 10, or drip chamber 9 may be further combined with an infusion monitor for adjustment of drip rate. In addition, drip chamber 9 and/or the infusion monitor are secured to a securing stay rod or other such securing hardware with which the IV stand is equipped so as to prevent the entire weight of the infusion set from being borne by intravenous drip needle(s) and connector(s) 12 for connection with intravenous chip needle(s). Furthermore, an infusion pump may he further combined therewith for adjustment of drip rate. This is useful at times when flow rate is low due to use of filter bag(s) 11 or use of therapeutic infusion(s) having high viscosity. Even where heavy infusion monitor(s) and infusion pump(s) are used in combination, by freely rotating the region(s) below a plurality of three-way stopcocks 5 having primary tubing downstream branch connectors 5b equipped with mechanisms that connect in such fashion as to permit rotation about primary tubing as axis or axes which are arranged at various locations in an infusion set in accordance with the present invention, adjustment of balance and elimination of torsion at tubing can be easily carried out. As a result, no abnormal force will act on the intravenous drip needle, and the risk of occurrence of accidents in which the intravenous drip needle becomes detached or there is leakage of liquid will be greatly reduced.

Furthermore, at the first member, with regard to the possibility that there may be a plurality of sets of secondary tubing due to presence of a plurality of linked three-way stopcocks 5, it is necessary that adequate attention be paid so as not to confuse the order of use of secondary tubing. In particular, where an anticancer agent or other such drug contraindicated for mixture with different drugs(s) is being used, the secondary tubing set connected to the downstream three-way stopcock is used first, and the secondary tubing set(s) connected to three-way stopcock(s) upstream therefrom is/are used thereafter in sequence,

### WORKING EXAMPLES

Indicated below are working examples of manufacture and use of infusion sets in accordance with the present invention. The present invention is not to be limited in any way by these descriptions.

Members for constructing an infusion set in accordance with the present invention were prepared in the form of a cap for installation on a spike; a spike; a three-way stopcock; a male connector; a drip chamber; a roller clamp; a connector for connection with an intravenous drip needle; infusion tubing; a shutoff clamp for pressing on and opening and/or closing flow path(s) within infusion tubing; an optional filter bag; a cap, for installation on a connector for connection with an intravenous drip needle, arranged at which there was an opening configured so as to not allow passage therethrough of solid or liquid but so as to allow passage therethrough of gas at the interior of the cap, a hydrophobic filter being arranged at a location inward from where the tip of the connector for connection with the intravenous drip needle was inserted at the interior of the cap by way of an insertion port for a connector for connection with an intravenous drip needle, and a lid at the exterior of said opening for closing, said opening; a connector for connection with a three-way stopcock; and a cap, for installation on a connector for connection with a three-way stopcock, arranged at which there was an opening not allowing passage therethrough of solid or liquid but allowing passage therethrough of gas, a hydrophobic filter being arranged at a location inward from where the tip of the connector for connection with the three-way stopcock was inserted, at the interior of the cap by way of an insertion port for a connector that connects with said three-way stopcock, and a lid at the exterior of said opening for closing said opening.

The foregoing members were connected to form a first member and second member(s) in the form of single integral units as described at the section entitled. Configuration and Constitution of Infusion Set of Present Invention and the section entitled Manufacturing Operations. In addition, caps for installation on connectors for connection with three-way stopcocks provided with lids and caps for installation on connectors for connection with intravenous drip needles provided with lids were definitively installed thereon, lids of the respective caps were opened, all shutoff clamps were placed in their open states, levers of three-way stopcocks by way of which the second member(s) were connected and three-way stopcocks at which there were no spikes, infusion tubing, or the like connected at secondary tubing branches for use as emergency ports were moved to positions permitting flow to/front primary tubing upstream branches and primary tubing downstream branches but not permitting flow to/from secondary tubing branches, and roller clamp 3 was placed in its open state. As shown in FIG. 1, an infusion set comprising a first member and four second members was manufactured as Working Example 1.

As Working Example 2, an infusion set which had three three-way stopcocks at a first member, tape respectively colored, in order from the bottom, red, white, and yellow being respectively attached thereto so as to cause these to be recognizable, and which furthermore had second members corresponding thereto, to which tape respectively colored, in order, red, white, and yellow was respectively attached so as to cause these to be recognizable, but which in other respects was identical to Working Example 1, was manufactured

As shown in FIG. 11, at Working Example 3, a package was prepared at which displayed on the surface thereof were illustrations that were images of tulips, the frontmost flower being colored red, the flower behind and overlapped by that one being colored white, and the backmost flower further overlapped by that one being colored yellow, the first member and the second members of the infusion set of Working Example 2 being sterilized and sealed therewithin to produce an infusion set.

As Comparative Example 1, an infusion set in which, instead of the caps having lids and hydrophobic filters employed at Working Example 1, caps were employed that differed only with respect to the fact that they were not provided with lids for closing openings, but which in other respects was identical to Working Example 1, was manufactured.

Two infusion containers having sealed therewithin physiological saline solution were suspended from an IV stand, the spikes respectively disposed at the upstream-most locations at the first member and the second member of the infusion set were made to pierce the rubber seals of said infusion containers, and priming operations were initiated. Testing was respectively carried out using the respective infusion sets at the respective Working Examples and at the respective Comparative Examples.

As a result, regardless of which infusion set was used, it was observed that priming of the infusion set could be completed as a result of only a single operation in which a spike was made to pierce the seal of an infusion container, the interiors of the first member and the second member(s) of the infusion set being filled with physiological saline solution, with only the air at the interior of the infusion set that was displaced by said physiological saline solution being exhausted by way of hydrophobic filters respectively provided at caps for installation on connectors for connection with three-way stopcocks and caps for installation on connectors for connection with intravenous drip needles. There was no leakage of physiological saline solution from any of the connections between/among the various members.

Next, following completion of priming, the lids for closing the openings at the caps, at which hydrophobic filters were disposed, installed at the first member and the second member(s) of the foregoing respective infusion sets were closed. Note that there were no lids on the caps at Comparative Example 1. In addition, shutoff clamps disposed at the respective sets of infusion tubing were pinched together, and the roller clamp was moved to its closed position, to close off the respective sets of infusion tubing.

Caps, at which hydrophobic filters were disposed, installed at the respective Working Examples and Comparative Example were removed therefrom

As a result, because the lids were closed at the caps in the respective Working Examples, air did not pass through the hydrophobic filters to reenter the cap interiors, and as a result of the action of surface tension due to the physiological saline solution and atmospheric pressure from the openings at the caps, physiological saline solution at regions peripheral to the hydrophobic filters at the interior did not spill out from the insertion ports at the caps. Furthermore, physiological saline solution did not drip out of the connectors for connection with intravenous drip needles or the connectors for connection with intravenous drip needles after they were detached.

However, at Comparative Example 1, which did not have lids, following removal of the caps from the connectors for connection with intravenous drip needles and the connectors for connection with intravenous drip needles, air did pass through the hydrophobic filters provided at those caps to reenter the cap interiors, and physiological saline solution that had remained at the cap interiors leaked out via the insertion ports for the connectors for connection with intravenous drip needles and the connectors for connection with intravenous drip needles at the caps and spilled out to the exterior.

Furthermore, in the infusion sets at the working examples of the invention under application, because secondary member(s) constituting instant tubing were not initially connected to the first member constituting primary tubing in the infusion set, there being no unwanted infusion tubing or the like hanging down about the periphery of the first member, it was possible to easily gain access to more than enough space in which to carry out procedures, and it was possible for preparatory procedures at the infusion set to proceed in satisfactory fashion. Furthermore, with regard to the second member(s), because these were connected from the bottom one-at-a-time in order of use, there was no inconvenience due to presence of unwanted instant tubing such as can occur when using a conventional infusion set, as it was the case for unconnected locations at secondary member(s) constituting instant tubing as well that it was similarly possible to easily gain access to more than enough space in which to carry out procedures, and it was found that it was possible to safely and definitively carry out preparatory procedures at the infusion set and also the intravenous drip procedures employing the infusion set that took place thereafter.

When connecting the first member and the second member(s), air present at locations at which connectors were mutually connected was assimilated by locations at which air which was present at the drip chamber.

At the infusion set of Working Example 2, because portions of the members were colored, it was possible with just a glance to recognize the respective sets of secondary tubing that were to be connected with the respective members and the spikes at the far ends thereof.

Furthermore, at the infusion set of Working Example 3, the illustrations depicted on the surface of the package immediately caught the eye, and it was possible to effortlessly recognize that red, white, and yellow coloring was employed in order from the frontmost depicted image to the images that were depicted therebehind. In addition, when the package was opened, it was possible with just a glance to recognize that portions of the members making up the sets of secondary tubing in the infusion set that was sealed therewithin were colored with the same colors.

### INDUSTRIAL UTILITY

In accordance with the means of the present invention, priming and backpriming operations can be carried out as a result of only a single operation in which a spike is made to pierce the seal of an infusion container containing physiological saline solution, and, there being no unintentional leakage of liquid while operations are being carried out or after operations have been carried out, it is possible to drastically reduce the risk of occurrence of damage to equipment and contamination of the hospital room interior, and the physiological saline solution can also be utilized in effective fashion during any medical treatment that may take place thereafter. Furthermore, it is possible to provide a novel infusion set that makes it possible to easily perform operations for alleviation of twisting and operations for adjustment of position of infusion tubing and/or infusion container(s) while maintaining the stability of the infusion set, and to prevent bodily pain to patients and/or leakage of liquid that might otherwise occur due to twisting and/or tension acting on the infusion set and/or intravenous drip needle(s) while lowering the risk of occurrence of accidents in which the IV stand from which the infusion set is suspended falls over. Moreover, it is possible to provide a novel infusion set that makes it possible to prevent occurrence of situations in which confusion as to order of use of a plurality of spikes provided at air infusion set when installing infusion container(s) causes error in the order in which infusions are to be administered, or in which infusion(s) containing different drug(s) become mixed up among multiple pieces of infusion tubing when there has been a change in set(s) of infusion(s) at the infusion set, and other accidents such as may occur due to human error, and that moreover makes it possible to easily establish standard procedures for use.

### EXPLANATION OF REFERENCE NUMERALS

1 Cap for installation on spike
2 Spike
3 Shutoff clamp
4 Infusion tubing
5 Three-way stopcock
5a Primary tubing upstream branch connector
5b Primary tubing downstream branch connector
5c Secondary tubing branch connector (needle-less port)
5d Lever
6 Cap fore installation on connector for connection with three-way stopcock
6a Insertion port for connector for connection with three-way stopcock
6b Hydrophobic filter
6c Opening
6d Lid
6e Connector for connection with three-way stopcock
7 Cap fore installation on connector for connection with intravenous drip needle
7a Insertion port for connector for connection with intravenous drip needle 7b Hydrophobic filter
7c Opening
7d Lid
8 Male connector
9 Drip chamber
10 Roller clamp
11 Filler bag
12 Connector for connection with intravenous drip needle
13 Infusion container
14 Physiological saline solution
15 Package
16 Illustration

1. An infusion set characterized in that
a first member constituting primary tubing in the infusion set has a spike; an optional spike cap; a three-way stopcock; a male connector; a drip chamber; a roller clamp; a connector for connection with an intravenous drip needle; a cap, for installation on the connector for connection with the intravenous drip needle, arranged at which there is an opening not allowing passage therethrough of solid or liquid but allowing passage therethrough of gas, a hydrophobic filter being arranged at a location inward from where a tip of the connector for connection with the intravenous drip needle is inserted at the interior of the cap by way of an insertion port for the connector for connection with said intravenous drip needle, a lid for closing said opening being arranged at the exterior of said opening; infusion tubing; a shutoff clamp for pressing on and opening and/or closing a flow path within the infusion tubing; and an optional filter bag;
a second member constituting secondary tubing in the infusion set has a spike; an optional spike cap; infusion tubing; a shutoff clamp for pressing on and opening and/or closing a flow path within the infusion tubing; a connector for connection with a three-way stopcock; and a cap, for installation on the connector for connection with the three-way stopcock, arranged at which there is an opening not allowing passage therethrough of solid or liquid but allowing passage therethrough of gas, a hydrophobic filter being arranged at a location inward from where a tip of the connector for connection with the three-way stopcock is inserted at the interior of the cap by way of an insertion port for the connector for connection with said three-way stopcock, a lid for closing said opening being arranged at the exterior of said opening;
wherein, prior to use of the infusion set, the first member constituting the primary tubing and the second member constituting the secondary tubing in the infusion set are in a mutually separate state; but during use thereof, a secondary tubing branch connector at the three-way stopcock of the first member constituting the primary tubing in the infusion set and a connector that connects with the three-way, stopcock at the second member constituting the secondary tubing in the infusion set are connected, causing the first member constituting the primary tubing and the second member constituting the secondary tubing in the infusion set to be in the form of a single integral unit;
wherein a spike is provided at upstream-most location in the first member constituting the primary tubing of the infusion set, said spike being optionally covered with a spike cap, and said spike being connected to one end of infusion tubing equipped with a shutoff clamp for pressing on and opening and/or closing a flow path within infusion tubing, the other end of said infusion tubing being connected to a primary tubing upstream branch connector of a three-way stopcock, and further connected to a primary tubing downstream branch connector of said three-way stopcock there is or are optionally one or more three-way stopcocks, and further connected to the primary tubing downstream branch connector of the three-way stopcock is one end of infusion tubing equipped with a shutoff clamp for pressing on and opening and/or closing an infusion tubing flow path, the other end of said infusion tubing being connected to a drip chamber, said drip chamber being connected to one end of infusion tubing on which a roller clamp is installed, the other end of said infusion tubing being connected to a primary tubing upstream branch connector of a three-way stopcock, a primary tubing downstream branch connector of said three-way stopcock being connected to one end of infusion tubing, the other end of said infusion tubing being optionally connected to a filter bag and infusion tubing downstream therefrom, and the other end of said infusion tubing being further connected to a primary tubing upstream branch connector of a three-way stopcock, a primary tubing downstream branch connector of said three-way stopcock being connected to one end of infusion tubing, the other end of said infusion tubing being connected to a connector for connection with an intravenous drip needle, said connector for connection with an intravenous drip needle being covered with a cap, for installation on the connector for connection with the intravenous drip needle, arranged at which there is an opening not allowing passage therethrough of solid or liquid but allowing passage therethrough of gas, a hydrophobic filter being arranged at a location inward from where a tip of the connector for connection with the intravenous drip needle is inserted at the interior of the cap by way of an insertion port for the connector for connection with the intravenous drip needle, a lid for closing said opening being arranged at the exterior of said opening; and
wherein a spike is provided at an upstream-most location in the second member constituting the secondary tubing of the infusion set, said spike being optionally covered with a spike cap, and said spike being connected to one end of infusion tubing equipped with a shutoff clamp for pressing on and opening and/or closing a flow path within infusion tubing, the other end of said infusion tubing being connected to a connector for connection with a three-way stopcock, the connector for connection with said three-way stopcock being covered with a cap, for installation on the connector for connection with the three-way stopcock, arranged at which there is an opening not allowing passage therethrough of solid or liquid but allowing passage therethrough of gas, a hydrophobic filter being arranged at a location inward from where a tip of the connector for connection with the three-way stopcock is inserted at the interior of the cap by way of an insertion port for the connector for connection with said three-way stopcock, a lid for closing said opening being arranged at the exterior of said opening.

2. The infusion set according to claim 1 characterized in that the aforesaid three-way stopcock at which infusion tubing is connected to a secondary tubing branch connector, said infusion tubing, a shutoff clamp linked thereto, and the cap are grouped together as a single group such that one or more thereamong has a location of the same color; and where there are a plurality of three-way stopcocks at which spikes are connected to secondary tubing, groups belonging to adjacent three-way stopcocks are made to have locations of respectively different colors; and said infusion tubing furthermore optionally has colored tape of the same color applied thereon; and any among the three-way stopcock, infusion tubing, shutoff clamp, spike, cap for installation on the spike, and colored tape is furthermore optionally in a state such that a number indicating order of administration is displayed thereon.

3. The infusion set according to claim 2 characterized in that the aforesaid colored infusion set is further sealed within a package at which displayed in mutually offset and partially overlapping fashion on the surface thereof are a plurality of illustrations, the respective illustrations being respectively colored so as to have the same colors as the coloration given to groups within the infusion set sealed therewithin that comprise the aforesaid three-way stopcocks at which infusion tubing is connected to secondary tubing branch connectors, said infusion tubing, shutoff clamps linked thereto, and the caps.

4. The infusion set according to claim 3 characterized in that numbers indicating order of administration are further displayed at the aforesaid illustrations.
